(19) United States
(12) Patent Application Publication
(10) Pub. No.: US 2023/0279600 A1
(43) Pub. Date: Sep. 7, 2023

(54) SYSTEM FOR AUTOMATICALLY WASHING AND DRYING A REUSABLE DIAPER

(71) Applicant: Edamame Technology LLC, San Francisco, CA (US)

(72) Inventors: Jonathan Palley, San Francisco, CA (US); Zhao Zhao, San Francisco, CA (US)

(21) Appl. No.: 18/116,231

(22) Filed: Mar. 1, 2023

Related U.S. Application Data
(60) Provisional application No. 63/315,464, filed on Mar. 1, 2022.

(51) Int. Cl. D06F 9/00; D06F 18/00; D06F 29/00; D06F 39/08; D06F 39/02; D06F 39/04; D06F 33/54; D06F 33/62; D06F 33/56; D06F 33/58; D06F 33/68; D06F 58/26; D06F 34/20; A46B 13/00; A46B 13/02; A46B 15/00; D06F 39/14; D06F 34/18; D06F 33/63; D06F 34/26; A61F 13/84; A61F 13/49

(52) U.S. Cl. CPC D06F 9/00; A46B 13/001; A46B 13/02; A46B 15/0004; A61F 13/49003; A61F 13/84; D06F 18/00; D06F 29/00; D06F 33/54; D06F 33/56; D06F 33/58; D06F 33/62; D06F 33/63; D06F 33/68; D06F 34/18; D06F 34/20; D06F 34/26; D06F 39/022; D06F 39/04; D06F 39/085; D06F 39/088; D06F 39/14; D06F 58/266; D06F 2105/58

(57) ABSTRACT

A system of washing and drying a reusable diaper including a housing defining a wash cavity configured to drain into a toilet bowl, reservoirs for water and detergent, a rack to retain the reusable diaper, a spray nozzle oriented toward the rack. Additionally, the system includes a brush head configured to dislodge waste, a fluid distribution system configured to distribute water from a water supply and detergent from the detergent reservoir, onto the reusable diaper via the spray nozzle, and a drying element configured to dry the reusable diaper retained on the rack. The system can include various configurations of frames to support the housing and allow access to a household water supply.

### CROSS-REFERENCE TO RELATED APPLICATIONS

This application claims the benefit of U.S. Provisional Application No. 63/315,464, filed on 01-MAR-2022, which is incorporated herein in its entirety by this reference.

### TECHNICAL FIELD

This invention relates generally to the field of automatic washing of articles of clothing and more specifically to a new and useful method for automatically washing and drying a reusable diaper in the field of automatic washing of articles of clothing.

### BRIEF DESCRIPTION OF THE FIGURES

FIGS. 1A and 1B are schematic representations of a first system;

FIG. 2 is a schematic representation of a variation of the first system;

FIG. 3 is a schematic representation of a variation of the first system; and

FIG. 4 is a schematic representation of variations of the first system.

FIG. 5 is a schematic representation of a variation of the first system;

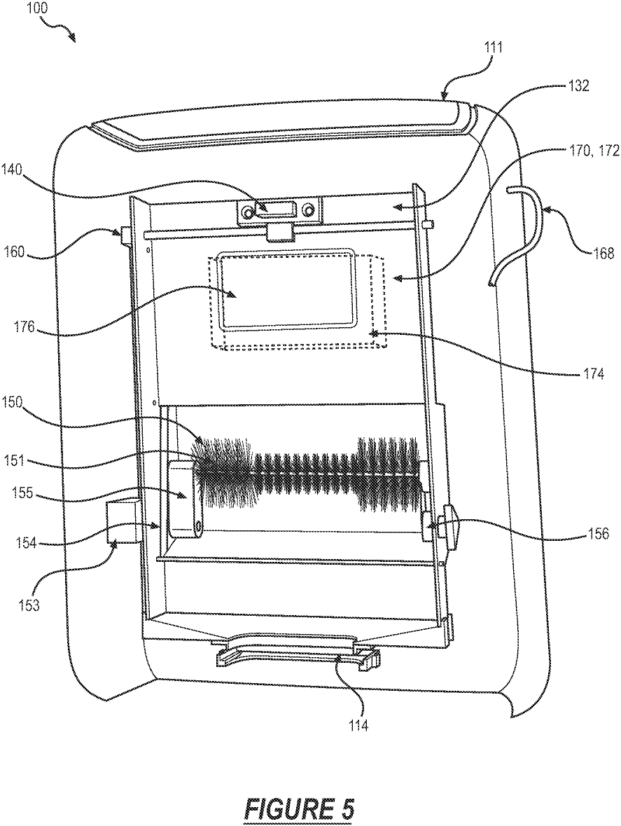

FIG. 6 is a schematic representation of a variation of the first system;

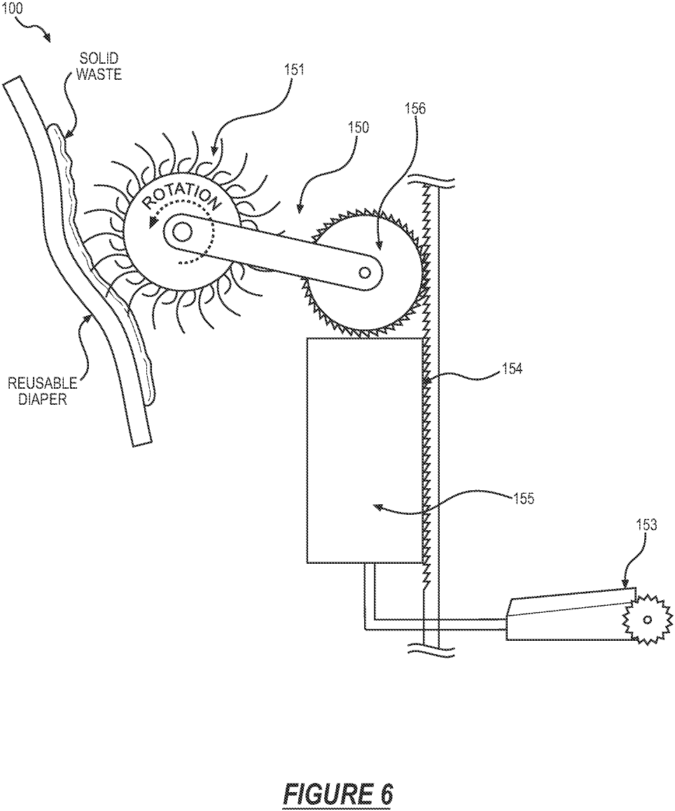

FIG. 7 is a block diagram of a variation of the first system.

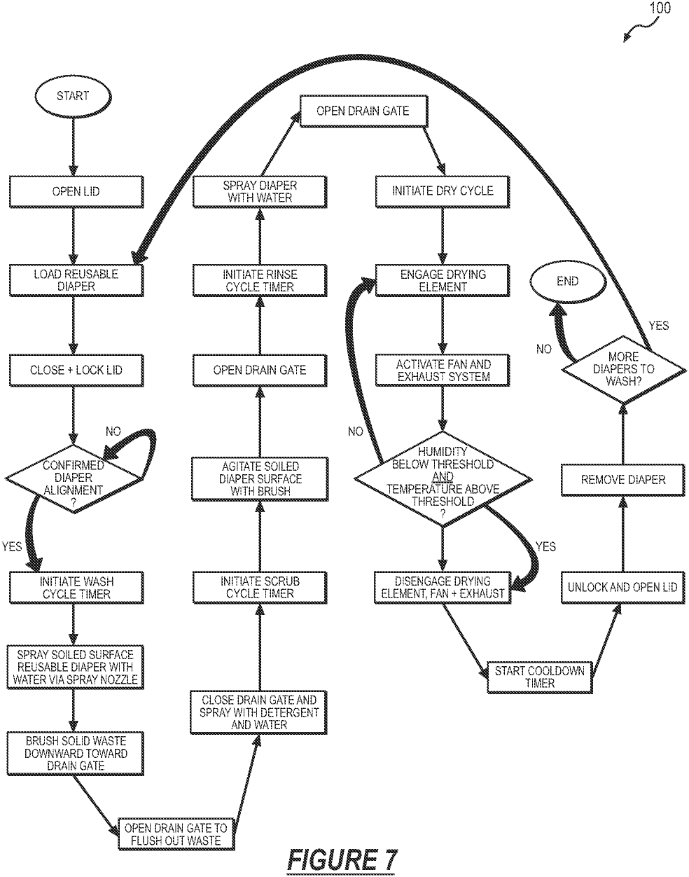

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. System

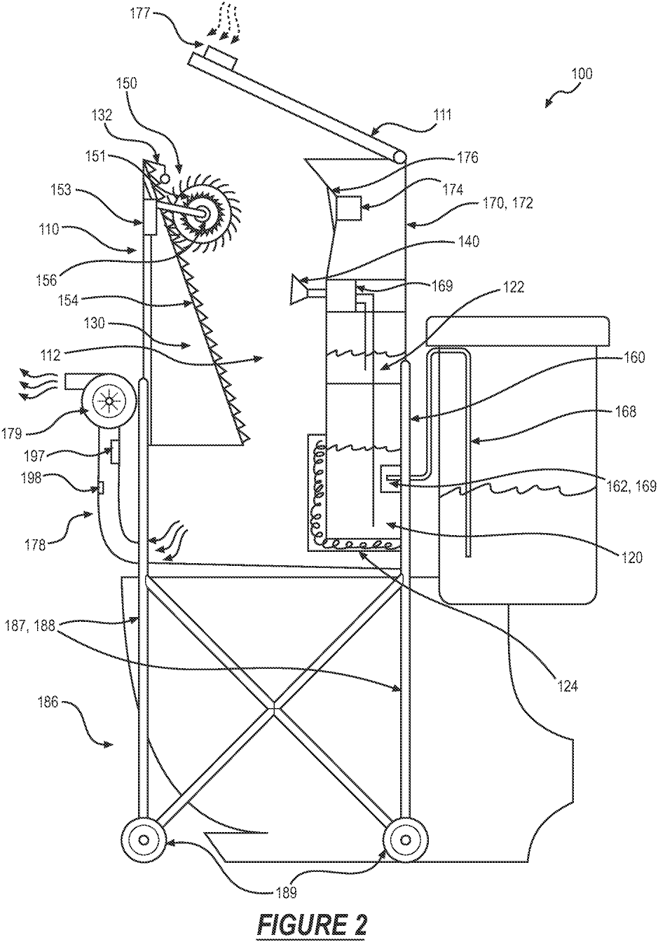

As shown in FIG. 2, a System 100 includes: a housing 110; a water reservoir 120; a detergent reservoir 122; a rack 130; a spray nozzle 140; a brush assembly 150; a fluid distribution system 160; and a drying element 170.

The housing 110 defines a wash cavity 112 and a drain gate 114 configured to face a toilet bowl.

The water reservoir 120 is arranged within the housing 110 and configured to store a first volume of water.

The detergent reservoir 122 is arranged within the housing 110 and configured to store a second volume of dilute detergent.

The rack 130 is arranged within the wash cavity 112 and configured to support and retain a reusable diaper during a processing cycle.

The spray nozzle 140 is arranged within the wash cavity 112 and configured to spray water, from the water reservoir 120, toward the reusable diaper retained on the rack 130.

The brush assembly 150 is arranged within the wash cavity 112, includes a brush head 151, and is configured to sweep the brush head 151 across the reusable diaper retained on the rack 130.

The fluid distribution system 160 is arranged within the housing 110 and configured to: distribute water from a water supply into the water reservoir 120; distribute water from the water reservoir 120 to the spray nozzle 140; distribute detergent from the detergent reservoir 122 onto the reusable diaper retained on the rack 130; and release soiled water into the toilet bowl via the drain gate 114.

The drying element 170: is arranged within the housing 110; and is configured to dry the reusable diaper retained on the rack 130.

One variation of the system 100 includes a housing 110 defining a wash cavity 112 and a drain gate 114 configured to face a toilet bowl. The system further includes a water reservoir 120 arranged within the housing 110 and configured store a first volume of water. The system also includes a detergent reservoir 122 arranged within the housing 110 and configured to store a second volume of dilute detergent. Additionally, the system includes a rack 130 arranged within the wash cavity 112 and configured to support and retain the reusable diaper during a processing cycle. The system includes a spray nozzle 140 arranged within the wash cavity 112 and configured to spray water, from the water reservoir 120, toward the reusable diaper retained on the rack 130. The system also includes a brush assembly 150 including a brush head 151, arranged within the wash cavity 112, and configured to sweep the brush head 151 across the reusable diaper retained on the rack 130. The system additionally includes a fluid distribution system 160 arranged within the housing 110 and configured to: distribute water from a water supply into the water reservoir 120; distribute water from the water reservoir 120 to the spray nozzle 140; distribute detergent from the detergent reservoir 122 onto the reusable diaper retained on the rack 130; and release soiled water into the toilet bowl via the drain gate 114. Further, the system includes a drying element 170 arranged within the housing 110 and configured to dry the reusable diaper retained on the rack 130.

### 1.1 Wall-Mounted System

Another variation of the system 100 includes a housing 110 defining a wash cavity 112 and a drain gate 114 configured to face a toilet bowl. The system includes a stationary frame 180 coupled to the housing 110, including a set of vertical supports vertically positioned on a left and a right side of a toilet, and configured to support a full weight of the housing 110 and mount to a wall. The system further includes a water reservoir 120 arranged within the housing 110 and configured to store a first volume of water. The system also includes a detergent reservoir 122 arranged within the housing 110 and configured to store a second volume of dilute detergent. Additionally, the system includes a rack 130 arranged within the wash cavity 112 and configured to support and retain the reusable diaper during a processing cycle. The system includes a spray nozzle 140 arranged within the wash cavity 112 and configured to spray water, from the water reservoir 120, toward the reusable diaper retained on the rack 130. The system also includes a brush assembly 150 including a brush head 151, arranged within the wash cavity 112, and configured to sweep the brush head 151 across the reusable diaper retained on the rack 130. The system additionally includes a fluid distribution system 160 arranged within the housing 110 and configured to: distribute water from a water supply into the water reservoir 120; distribute water from the water reservoir 120 to the spray nozzle 140; distribute detergent from the detergent reservoir 122 onto the reusable diaper retained on the rack 130; and release soiled water into the toilet bowl via the drain gate 114. The system also includes a valve 164 fluidly coupled to a household waterline, the household waterline configured to supply the fluid distribution system 160 with the first volume of water, and the valve 164 configured to: in an open position, allow flow of the first volume of water to the water reservoir and in a closed position, restrict flow of the first volume of water to the water reservoir. Further, the system includes a drying element 170 arranged within the housing 110 and configured to dry the reusable diaper retained on the rack 130.

### 1.2 Mobile System

Yet another variation of the system 100 includes a housing 110 defining a wash cavity 112 and a drain gate 114 configured to face a toilet bowl. The system includes a mobile frame 186 configured to position the housing 110 over the opening of the toilet bowl. The mobile frame 186 includes a set of vertical supports, vertically positioned on a left side and a right side of a toilet, and a set of casters 189 coupled to the set of vertical supports. The mobile frame 186 is configured to allow movement of the system between a first toilet and a second toilet. The system further includes a water reservoir 120 arranged within the housing 110 and configured to store a first volume of water. The system also includes a detergent reservoir 122 arranged within the housing 110 and configured to store a second volume of dilute detergent. Additionally, the system includes a rack 130 arranged within the wash cavity 112 and configured to support and retain the reusable diaper during a processing cycle. The system includes a spray nozzle 140 arranged within the wash cavity 112 and configured to spray water, from the water reservoir 120, toward the reusable diaper retained on the rack 130. The system also includes a brush assembly 150 including a brush head 151, arranged within the wash cavity 112, and configured to sweep the brush head 151 across the reusable diaper retained on the rack 130. The system additionally includes a fluid distribution system 160 arranged within the housing 110 including a set of pumps 169 and configured to: draw water, via a hose 168, from a toilet tank into the water reservoir 120; distribute water from the water reservoir 120 to the spray nozzle 140; and distribute detergent from the detergent reservoir 122 onto the reusable diaper retained on the rack 130. Further, the system includes a drying element 170 arranged within the housing 110 and configured to dry the reusable diaper retained on the rack 130.

### 2. Applications

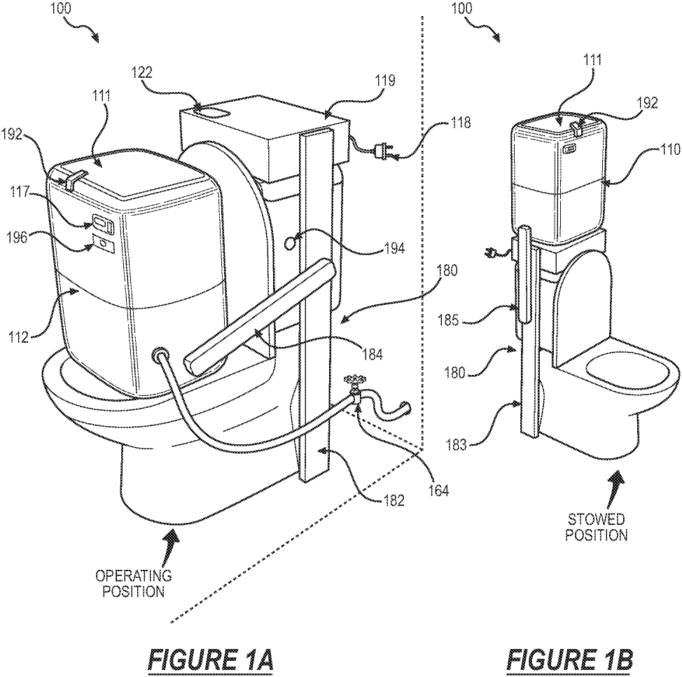

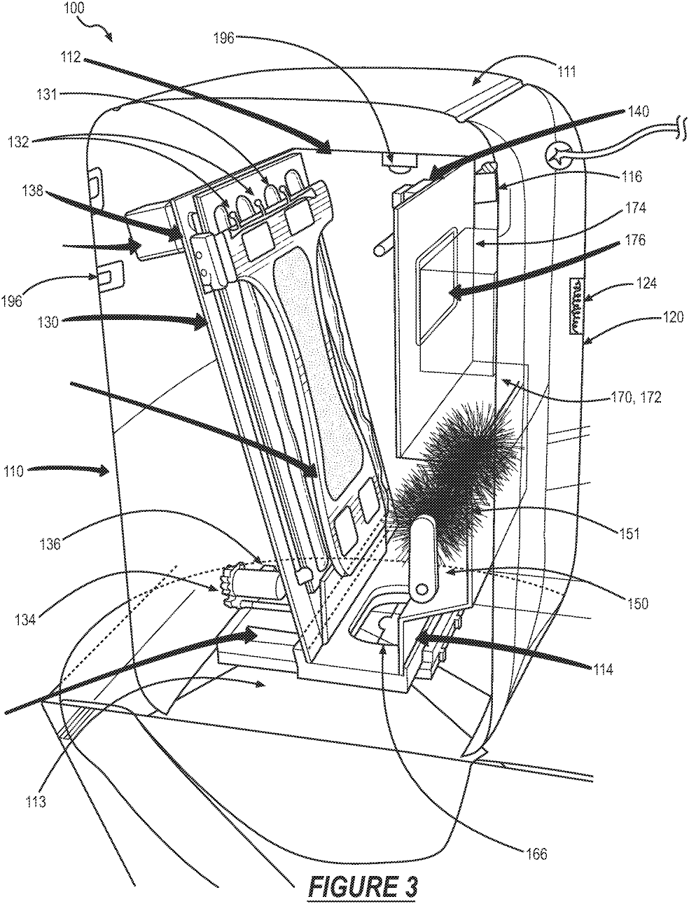

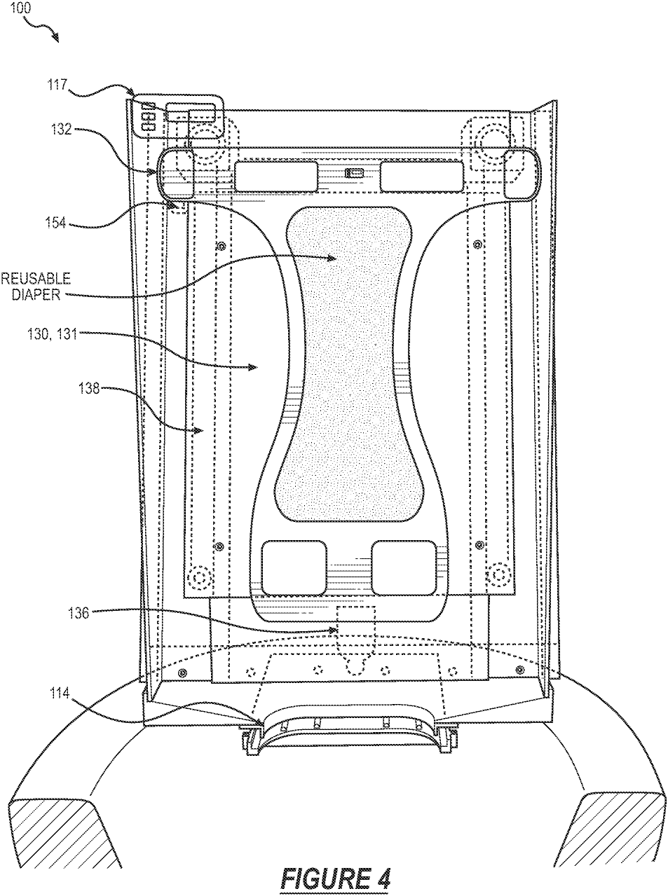

Generally, as shown in FIGS. 1-4, the system 100 defines a housing 110 configured to: receive a single soiled reusable diaper; remove waste matter from the single reusable diaper using water, detergent, and agitation; rinse the reusable diaper; evacuate waste and water-detergent solution into a toilet bowl; and dry the reusable diaper using a drying element 170. Generally, a frame (e.g., mobile, stationary) mounted to the housing 110 supports the weight of the housing 110 and the housing 110 can transition from a stowed position to an operating position and back again, thereby enabling a user to install the system 100 proximal an existing toilet (e.g., in the user’s home), operate the housing 110 in the operating position, and stow the housing 110 in the stowed position when not in use. The frame counterbalances the weight of the housing 110 such that the user may exert minimal force to transition the housing 110 from the stowed position to the operating position, and vice-versa. For example, the system 100 can include the counterbalance opposite the housing 110 to enable a user holding an infant to transition the housing 110 between the operating and stored positions with a single hand.

The housing 110 is configured to receive water from a water supply in the user’s home and configured to connect to an existing electrical outlet proximal the toilet. The housing 110 also evacuates waste into the toilet bowl, an existing structure configured to effectively remove bodily waste from the user’s home. The features of the system 100 cooperate to conveniently exploit existing features of a user’s home to create an effective solution to removing waste from a reusable diaper.

The system 100 performs a unique processing cycle designed to effectively remove waste from a single reusable diaper. The processing cycle can include subprocesses such as: an initial rinse cycle, a wash cycle; a scrub cycle; a rinse cycle; and a dry cycle. Prior to execution of the processing cycle, the user affixes the reusable diaper to a rack 130, via an attachment feature (e.g., set of clips 132, pins, clamps, retention ports), within the wash cavity 112 of the housing 110, such that the soiled side of the reusable diaper is exposed to a spray nozzle 140 mounted in the wash cavity 112, opposite the rack 130. The spray nozzle 140 performs an initial rinse cycle to dislodge solid waste from the reusable diaper. To perform the initial rinse cycle, the drain gate 114 of the housing 110 is opened, exposing the wash cavity 112 to the toilet bowl below. The spray nozzle 140 releases a stream of water to impact the soiled side of the reusable diaper. The stream of water dislodges solid waste.

After the initial rinse cycle, the wash cycle initiates. The drain gate 114 of the housing 110 closes, and a detergent reservoir 122 arranged within the housing 110 dispenses a volume of detergent into the water supply line to the spray nozzle 140 to create a water-detergent solution which passes through the spray nozzle 140 and introduces the volume of detergent into the wash cavity 112 during the wash cycle. The water-detergent solution impacts the reusable diaper and drains to the bottom portion of the wash cavity 113 and collects in a sump. A sump pump draws water-detergent solution from the sump and pumps it through the spray nozzle 140, via the fluid distribution system 160, and back into the wash cavity 112. The sump includes a sump filter, reducing the size and number of solid waste particles drawn in by the pump. By re-circulating the water-detergent solution during the wash cycle, the system 100 reduces a total volume of water required to clean a reusable diaper.

During the scrub cycle, succeeding the initial rinse cycle, the rack 130 can translate toward the bottom portion of the wash cavity 113, into the volume of water-detergent solution collected in the bottom portion of the wash cavity 113. The brush head 151 rotates and a brush assembly 150 (e.g., articulating, translating) brings the brush head 151 into contact with the soiled side of the reusable diaper. In one variation, the rack 130 can raster from a first position below a brush head 151 to a second position above the brush head 151 along the linear track 138, the brush head 151 in contact with the reusable diaper retained by the rack 130. In another variation, the brush head 151 can raster from a first position above the rack 130 to a second position below the rack 130. Generally, the brush head 151 rotates such that the reusable diaper tensions against the set of clips 132 and waste dislodged from the reusable diaper is directed downward toward the bottom portion of the wash cavity 113 and into the water-detergent solution. In one variation, the brush head 151 is connected to a bi-directional motor and rotates in both directions during the scrub cycle to further agitate and scrub the reusable diaper. When the scrub cycle is complete, the rack 130 translates to bring the reusable diaper up and out of the water-detergent solution.

During a rinse cycle, succeeding the scrub cycle, the drain gate 114 opens to release the waste from the wash cavity 112 into the toilet bowl. The fluid distribution system 160 draws fresh water from the water reservoir 120, via a pump, and pumps it through the spray nozzle 140. The spray nozzle 140 pivots up and down to rinse the reusable diaper as the waste passes into the toilet bowl. After a preselected duration, the pump ceases to pump water to the spray nozzle 140 and the rinse cycle terminates.

During a drying cycle, succeeding the rinse cycle, the drain gate 114 closes to maintain a watertight seal and electromagnetic shielding. The system 100 activates the drying element 170, a microwave, to emit microwaves directed at the reusable diaper into the wash cavity 112, for a preselected duration of time. More specifically, water molecules in the reusable diaper absorb microwave energy - emitted by the drying element 170 -and evaporate from the reusable diaper. In one variation, the system 100 further includes a fan 179 that circulates air into the wash cavity 112 and a vent 177 that releases steam from the wash cavity 112 to the ambient surroundings during this drying cycle. In another variation, sensors (e.g., humidity, temperature, gas, infrared) in electronic communication with a controller 116 derive the diaper to be dry, based on a crying cycle duration, an ambient temperature, an ambient humidity, a surface temperature of the reusable diaper, etc. In particular, the microwaves emitted by the drying element 170 vaporize water molecules trapped in the reusable diaper, drying the reusable diaper.

At the conclusion of the drying cycle, the housing 110 emits an audible and/or visual signal to inform the user the processing cycle is complete. The user then removes the reusable diaper from the wash cavity 112 and returns the housing 110 to the stowed position.

### 2.1 Wall-Mounted System

In one variation, the system 100 can further include a wall mount including a left vertical support 182 and a right vertical support 183, with arm members 184, 185 coupled to at least one vertical support on the left and right side of the housing 110, and pivotably attached to the respective sides of the housing 110. The wall mount supports the weight of the housing 110 and can be shifted to transition the housing 110 from a stowed position to an operating position and back again, thereby enabling the system 100 to execute in conjunction with an existing toilet, sink, tub, or shower (e.g., in the user’s home). More specifically, the system 100 is semi-permanently coupled (e.g., tee joint) to a pressurized water supply, such as a household waterline, and regulated by a valve 164 to selectively fill the water reservoir 120 for use in the processing cycle.

### 2.2 Mobile System

In another variation, the system 100, can further include a mobile frame 186 including a first set of vertical supports 187 and a second set of vertical supports 188, with arm members 184, 185 coupled to at least one vertical support on the left and right side of the housing 110, and pivotably attached to the respective sides of the housing 110. The wall mount supports the weight of the housing 110 and can be shifted to transition the housing 110 from a stowed position to an operating position and back again, thereby enabling the system 100 to execute in conjunction with an existing toilet, sink, tub, or shower. More specifically the mobile housing 110 also includes a set of casters 189 coupled to both the left and right sets of vertical support 182 183. The casters are configured to allow movement of the system 100 between a first site and a second site in the user’s home. The variation of system 100 also includes a hose 168 arranged in the water supply (e.g., a toilet tank) and a set of pumps 169 configured to pump water from the water supply into the water reservoir 120.

### 2.3 Direct Water Delivery System

In yet another variation, the system 100 can include a housing 110, a detergent reservoir 122, a rack 130, a spray nozzle 140, a brush assembly 150, a fluid distribution system 160, and a drying element 170. More specifically, the housing 110 can define a wash cavity 112 and a drain gate 114 configured to face a toilet bowl. The detergent reservoir 122 can be arranged within the housing 110 and configured to store a second volume of dilute detergent. The rack 130 can be arranged within the wash cavity 112 and configured to support and retain the reusable diaper during a processing cycle. Further, the spray nozzle 140 can be arranged in the wash cavity 112 and configured to spray water toward the reusable diaper, while the brush assembly 150 can be arranged in the wash cavity 112, include a brush head 151, and be configured to sweep the brush head 151. The fluid distribution system 160 can be arranged in the housing 110, include a set of pumps 169, and be configured to: draw water, via the hose 168, from the toilet tank to the spray nozzle 140 and distribute detergent from the detergent reservoir 122 onto the reusable diaper retained on the rack 130. The drying element 170 can be arranged within the housing 110 and configured to dry the reusable diaper retained on the rack 130.

In this variation, during the processing cycle, the controller 116 can actuate the set of pumps 169 (and/or valves 164) to spray water (e.g., unheated water) directly onto the reusable diaper via the spray nozzle 140. The controller 116 can then trigger the drain gate 114 to open, thereby releasing liquid and/or solid waste thus removed from the reusable diaper to flow from the wash cavity 112 into the toilet below. Upon expiration of a preset drain duration (e.g., seven seconds), the controller 116 triggers the drain gate 114 to close. The controller 116 then triggers the set of pumps 169 (and/or valves 164) to spray water and detergent onto the reusable diaper via the spray nozzle 140, such as a target volume of wash water and detergent. The water and detergent flow across and wet the reusable diaper; excess wash water and detergent collect in the base of the wash cavity 112 above the drain gate 114. The controller 116 then triggers the rack actuator 136 to drive the rack 130 and the reusable diaper downwardly toward the brush head 151 and the bottom portion of the wash cavity 113. As the rack 130 moves downwardly toward the base of the wash cavity 112, the controller 116 can rotate the brush head 151 in a forward direction to push the reusable diaper - between the rack 130 and the brush head 151 -downward against the rack 130, thereby tensioning the reusable diaper along the rack 130 and guiding the reusable diaper into the pool of wash liquid in the bottom portion of the wash cavity 113. With the reusable diaper thus predominantly (or fully) submerged in this volume of wash liquid, the controller 116 can then trigger the rack actuator 136 draw the rack 130 upwardly and while continuing to rotate the brush assembly 150 in the forward direction. The brush head 151 thus scrubs saturated surface of the reusable diaper, maintains tension on the reusable diaper as the rack 130 draws the reusable diaper out of the pool of wash liquid, and further removes waste from the reusable diaper. Once the rack 130 reaches its to start position, the controller 116 can trigger the drain gate 114 to open and thus release the wash liquid into the toilet bowl below.

The controller 116 can then repeat the foregoing process for one or more additional wash cycles (e.g., by default or in response to the user indicating a heavily-soiled reusable diaper via the user interface 117). Additionally or alternatively, the controller 116 can repeat this process solely with water (i.e., without detergent) in order to rinse the reusable diaper, such as in one or a series of rinse cycles.

The controller 116 can then actuate the drying element 170 (e.g., a microwave emitter 174) to heat the reusable diaper, kill or inactivate remnant microbes in the reusable diaper, drive moisture out of the reusable diaper, and thus sanitize and dry the reusable diaper. The system can therefore: wash and rinse the reusable diaper with small volumes of cool or otherwise unheated water; and sanitize the reusable diaper by heating with a microwave emitter 174.

### 3. System Components

Generally, the system 100 can include a housing 110 and a frame, the housing 110 attached to the frame, allowing the housing 110 to be transitioned between a stowed position and an operational position. The housing 110 can connect to an external water supply such as a household waterline or toilet tank, and to an external power source such as a wall outlet located proximal the toilet.

### 3.1 Housing

Generally, the housing 110 can include: a wash cavity 112, a water reservoir 120; a detergent reservoir 122; a fluid distribution system 160; a drying element 170; and a controller 116. The wash cavity 112 can include: a rack 130; a spray nozzle 140; and a brush assembly 150. The housing 110 can also include: a lid 111 configured to swing open and permit access to an interior of the wash cavity 112 and latch 192 (e.g., electromagnetic) closed to prevent unintended opening during the wash cycle and/or drying cycle; and a drain gate 114 configured to open and close to selectively release the contents of the wash cavity 112.

In one variation, a second housing 119, external to the housing 110, can contain the water reservoir 120, the detergent reservoir 122, and a power supply connection 118. The water reservoir 120 and the detergent reservoir 122 can connect to the fluid distribution system 160 and the spray nozzle 140, in the housing 110, via a fluid connection.

The housing 110 includes a water supply connection that connects a water supply, proximal the toilet, to the fluid distribution system 160. The fluid distribution system 160 draws fresh, clean water from the water supply, via the water supply connection into the water reservoir 120. The water supply can include a pressurized supply (e.g., household waterline) and utilize a valve 164 to selectively regulate flow, or a water supply (e.g., toilet tank) requiring a pump to draw a volume of water into the water reservoir 120.

Generally, the system includes a controller 116, in communication with the various sensors, electrical components, and electro-mechanical components within the housing 110. Generally, an electrical connection (e.g., power supply connection 118) connects the controller 116 to a power supply, such as a wall outlet proximal the toilet, and provides power to the system 100.

Generally, the bottom face of the housing 110: includes a drain gate 114, defines a face with a width larger than the inner diameter of a toilet bowl rim; and is configured to mate with a toilet bowl such that the housing 110 sits level on the toilet bowl rim and does not unseat from the toilet bowl rim while in use.

### 3.2 Frame

Generally, the frame: is configured to support the housing 110 in a stowed position above the tank of the toilet; allows the housing 110 to transition to an operating position proximal the bowl of the toilet for use; and transition back to the stowed position. The frame can include a left vertical support 182 positioned vertically on a left side of the toilet, in contact with the wall proximal the back of the toilet, and a right vertical support 183 similarly positioned on a right side of the toilet. The left and right vertical supports 183 can mount to the wall proximal the toilet. The frame further includes: a left arm member 184 coupled to the left vertical support 182, via a first spring hinge, and pivotably attached to a left side of the housing 110; and a right arm member 185 coupled to the right vertical support 183, via a second spring hinge, and pivotably attached to a right side of the housing 110. The pivoting attachments maintain the upright orientation of the housing 110 when the housing 110 transitions between a stowed position above the toilet tank, and an operating position proximal the toilet bowl. Additionally, the frame supports the housing 110 over the toilet bowl to prevent it from unseating from the toilet bowl while in use. Further, the frame supports and protects the water supply connection. In one variation, each of the arm members 184, 185 is a spring-loaded parallel linkage that counterbalances the weight of the housing 110 and maintains orientation of the housing 110 in the stowed position, the operating position, and in transition between positions. In another variation, the frame supports and protects the power supply connection 118 between the housing 110 and external power source.

### 3.2.1 Wall Mount Frame

In one implementation, the water supply includes a pressurized residential waterline fluidly coupled to the fluid distribution system 160 and configured to supply the fluid distribution system 160 with the first volume of water. In this implementation, the system 100 can include a stationary frame 180 including: a left vertical support 182 vertically positioned on a left side of the toilet; a right vertical support 183 vertically positioned on a right side of the toilet; a left arm member 184 pivotably attached to a left side of the housing 110 and coupled to the left vertical support 182; and a right arm member 185 pivotably attached to a right side of the housing 110 and coupled to the right vertical support 183. The stationary frame 180 configured to support the housing 110 in a stowed position above the tank of toilet and enable the housing 110 to transition to an operating position above the opening of the toilet bowl.

### 3.2.2 Mobile Frame

In one example, the frame can include a mobile frame 186 including: a first set of vertical supports 187 vertically positioned on a left side of the toilet; a second set of vertical supports 188 vertically positioned on the right side of the toilet; a left arm member 184 pivotably attached to the left side of the housing 110 and coupled to the first set of vertical supports 187; a right arm member 185 pivotably attached to the right side of the housing 110 and coupled to the second set of vertical supports 188; and a set of casters 189, coupled to the first set of vertical supports 187 and the second set of vertical supports 188, configured to allow movement of the system between a first toilet and a second toilet. The mobile frame 186 can be configured to support the housing 110 in a stowed position above the tank of toilet and enable the housing 110 to transition to an operating position above the opening of the toilet bowl.

### 3.3 Water Supply

Generally, the housing 110 is connected to the water supply (e.g., pressurized residential waterline) of a toilet, a sink, or a shower via a tee junction at the water supply valve 164 proximal the toilet, a sink, or a shower. The water supply connects to the fluid distribution system 160 in the housing 110. The fluid distribution system 160 draws water from the household water supply into the water reservoir 120 within the housing 110. In one variation, the household water supply connection is connected to an inlet valve, the inlet valve in turn connected to the water reservoir 120. In this variation, when the inlet valve is opened, the water pressure of the household water supply forces water into the reservoir. When the water reservoir 120 is full, the inlet valve automatically closes and ceases the flow of water from the household water supply into the water reservoir 120. In another variation, the water supply is a toilet tank connected to the water reservoir 120 via a pump (e.g., peristaltic, hydraulic, air, etc.) and a hose 168 (e.g., pipe, tube, channel) extending into the toilet tank.

In one example, the water supply can include a toilet tank and a hose 168, fluidly coupled to the water reservoir 120. The system 100 can further include a set of pumps 169 configured to: draw water, via the hose 168, from the toilet tank to the water reservoir 120; fill the water reservoir 120 with the first volume of water; and pump water from the water reservoir 120 to the spray nozzle 140.

### 3.4 Water Reservoir

Generally, the water reservoir 120 is arranged within the housing 110, configured to receive a volume of water via the fluid distribution system 160, and retain the volume of water. The water reservoir 120 retains a volume of water sufficient to complete at least one processing cycle. The fluid distribution system 160 connects the water reservoir 120 to the water supply and the spray nozzle 140. In one variation, a heating element 124 is configured to heat the volume of water stored in the water reservoir 120 to a wash temperature.

### 3.5 Detergent Reservoir

Generally, the detergent reservoir 122 is arranged within the housing 110 and configured to retain a volume of a detergent. In one variation, the detergent reservoir 122 can, via a pump (e.g., metering pump), introduce a volume of detergent into the connection between the water reservoir 120 to the spray nozzle 140 producing a dilute detergent. In another variation, the detergent reservoir 122 is connected to the water supply via the fluid distribution system 160 and receives both water and detergent, producing a dilute detergent in the detergent reservoir 122. Periodically, the detergent reservoir 122 receives a volume of detergent, to increase the volume of detergent retained within the detergent reservoir 122 beyond a threshold volume (i.e., the detergent reservoir 122 is periodically refilled with detergent). In one variation, a heating element 124 is configured to heat the volume of dilute detergent stored in the water reservoir 120 to a wash temperature.

In one variation, the detergent reservoir 122 is configured to accept individual detergent packages for a single processing cycle, such as a water-soluble pod or a single-use plastic container containing a dose of detergent for a single processing cycle. Further, in another variation, the detergent reservoir 122 stores a volume of detergent in solution with a volume of water, such as a total volume of dilute detergent in equal to a volume necessary to complete a single processing cycle.

In one implementation, the detergent reservoir 122 stores concentrated detergent. The fluid distribution system 160 includes: a water pump 162 plumbed to a sump transiently inserted into a water tank of the toilet; a spray nozzle 140 facing the rack 130; an internal water supply line extending from the water pump 162 to the spray nozzle 140; a detergent tap (or venturi) located along the internal water supply line; and a detergent pump 161 (or valve) configured to pump (or release) concentrated detergent from the detergent reservoir 122 into the internal water supply line where detergent mixes with water sprayed across the rack 130. Additionally or alternatively, in this implementation, the detergent reservoir 122 includes a cartridge receptacle configured to receive a replaceable detergent cartridge loaded with detergent.

However, the system 100 can receive and store concentrate or dilute powdered or liquid detergent (and/or soap) in any other volume or format and can distribute detergent (or soap) into the wash cavity 112 in any other way during a wash cycle.

### 3.6 Wash Cavity

Generally, the wash cavity 112 includes two openings: a lid 111 at an upper section of the wash cavity 112, configured to open and permit access to the interior of the wash cavity 112 when opened; and a drain gate 114 located at a bottom portion of the housing 110 configured to automatically open and close during a wash cycle to selectively retain or release water, detergent, and/or waste contained in the wash cavity 112.

In one variation, the wash cavity 112 is surrounded by microwave opaque material to contain any microwave radiation emitted into the wash cavity 112 and to shield any sensitive electronics or persons proximal the housing 110 during operation. The lid 111 and drain gate 114 include microwave opaque material sufficient to electromagnetically shield the interior of the wash cavity 112 during operation when the lid 111 and drain gate 114 are closed. The lid 111 includes a latch 192 to prevent inadvertent opening of the housing 110 and reduce the likelihood that microwave radiation (or water) will escape the interior of the wash cavity 112 during operation.

Generally, the wash cavity 112 is configured to retain the heat generated by the heating element 124. The wash cavity 112 is further configured to retain waste, detergent, and/or water until automatic release into the toilet bowl via the drain gate 114.

### 3.7 Rack

Generally, the rack 130 defines an inclined surface arranged within the wash cavity 112, opposite the heating element 124 and spray nozzle 140. In one variation, the rack 130 defines a perforated panel 131, is coupled to a set of clips 132, is engaged with a rack actuator 136 and a linear track 138, and is configured to retain a reusable diaper with the set of clips 132 and raster within the wash cavity 112, proximal the brush assembly 150, to maintain contact between the brush head 151 and the reusable diaper retained on the rack 130 by the set of clips 132.

In another variation, a translating carrier is configured to translate the rack 130 within the wash cavity 112 to lower the reusable diaper into the volume of washing liquid (e.g., detergent, water) that collects in the bottom portion of the wash cavity 113 during the wash cycle, and into contact with the brush assembly 150. The translating carrier is also configured to raise the reusable diaper out of the volume of washing liquid and stretch the reusable diaper flat along the rack 130 to enable more effective drying of the reusable diaper during the drying phase.

The rack 130 is mounted to the translating carrier and configured to support the set of clips 132. The rack 130 is arranged at the top edge of a washing area. The washing area defines: an area extending from the upper translation limit of the translating carrier proximal the lid 111 of the housing 110, to the bottom portion of the wash cavity 113 proximal the drain gate 114 of the housing 110; and from a left edge of the wash cavity 112 to a right edge of the wash cavity 112. The reusable diaper is retained on the rack 130, with the set of clips 132, such that the soiled side of the reusable diaper are arranged within the washing area facing the spray nozzle 140, opposite the rack 130.

In one implementation, the set of clips 132 is arranged on the rack 130. The reusable diaper is retained by the rack 130 - arranged in an upper section of the washing area - with the set of clips 132. The remainder of the reusable diaper is unconstrained but rests against the rack 130, the soiled side facing opposite the rack 130. The washing liquid is directed to the washing area to clean the reusable diaper during the wash cycle. Washing liquid collects in the bottom portion of the wash cavity 113 during the wash cycle. When the translating carrier lowers the reusable diaper into the volume of washing liquid collected in the bottom portion of the wash cavity 113, the reusable diaper deforms to contact a greater surface of the brush assembly 150.

### 3.8 Spray Nozzle

The spray nozzle 140 is arranged in the upper section of the wash cavity 112, opposite to and facing the rack 130. The spray nozzle 140 is configured to: release a water stream in a pattern such that the water stream can impact the whole of the reusable diaper; and to pivot in the vertical direction such that the water stream can dislodge solid waste, from the reusable diaper, and direct it toward the bottom portion of the wash cavity 113, and subsequently out of the wash cavity 112 when the drain gate 114 is open. In one variation, the spray nozzle 140 is configured to receive and spray a water-detergent solution (e.g., dilute detergent) without clogging. In one implementation, the spray nozzle 140 is a wide-angle spray nozzle configured to spray the whole of the reusable diaper simultaneously.

In one example, the system 100 can also include a second spray nozzle 142 arranged within the wash cavity 112. The spray nozzle 140 is configured to: during a wash cycle, spray detergent, from the detergent reservoir 122, onto the reusable diaper retained on the rack 130; and during a rinse cycle, spray water, from the water reservoir 120, onto the reusable diaper retained on the rack 130.

### 3.9 Brush Assembly

Generally, the brush assembly 150 includes a brush head 151. The brush head 151 can be mounted to an articulating brush support. The brush head 151 can further include a set of flexible silicone bristles on the face of a cylinder, mounted to an articulated support configured to bring the flexible bristles into contact with the reusable diaper and dislodge waste not previously dislodged by the initial water stream. The brush head 151 is connected to a brush actuator 153 and configured to rotate such that the brush head 151 rotates from the direction of the top portion of the wash cavity 112 toward the bottom portion of the wash cavity 113, thereby reducing the likelihood of projecting waste particles out of the water-detergent solution and into the upper section of the wash cavity 112 where they can become trapped. The downward scrubbing motion of the brush head 151 directs dislodged solid waste into the washing liquid and/or toward the drain gate 114, increasing the likelihood that waste particles become and remain suspended in the washing liquid, and will flow out of the wash cavity 112 when the washing liquid is released. Additionally, the motion of the brush head 151 agitates the water-detergent solution to homogenize the water-detergent solution and move detergent through the reusable diaper to interact with and emulsify waste adhered to and embedded in the reusable diaper.

In another variation, the brush head 151 is connected to a bi-directional motor, and thus the brush head 151 can rotate in both a clockwise and counterclockwise direction. A bi-directional scrubbing action is advantageous to dislodge waste particles that are strongly adhered to the reusable diaper.

The brush head 151 is mounted to an articulated brush support that can pivot to bring the brush head 151 into contact with the reusable diaper, the reusable diaper attached to the rack 130. The articulated brush head 151 support brings the brush head 151 into contact with the washing area to enable the brush head 151 to agitate the reusable diaper thereby dislodging waste from the reusable diaper attached to the rack 130.

In one example, the rack 130 defines a perforated panel 131 inclined within the wash cavity 112 and configured to locate a soiled side of the reusable diaper facing the spray nozzle 140 and the brush head 151. The rack 130 can include a set of clips 132 arranged on the rack 130 and configured to retain a top edge of the reusable diaper on the rack 130. In this example, the brush assembly 150 is configured to: traverse downwardly, proximal the rack 130, to scrub waste downward; maintain contact between the brush head 151 and the soiled side of the reusable diaper; and tension the diaper downwardly against the set of clips 132 during a scrub cycle in the processing cycle.

### 3.10 Drain Gate and Liquid Evacuation

Generally, the drain gate 114, arranged in the bottom portion of the wash cavity 113 automatically opens to release the liquid contents of the wash cavity 112, and automatically closes to seal the wash cavity 112.

When closed, the drain gate 114 maintains a watertight seal between the wash cavity 112 and the exterior of the housing 110. The drain gate 114 pivots downward toward the toilet bowl to release the liquid contents of the wash cavity 112 into the toilet bowl below. When the drain gate 114 opens, water, detergent, washing liquid, solid waste, and/or liquid waste are evacuated from the housing 110 via a combination of gravity and the force of the water stream from the spray nozzle 140 arranged in the upper section of the wash cavity 112.

In one variation, the drain gate 114 opens via a sliding motion. However, any suitable mechanism for repeatable opening and closing a valve 166 while maintaining a watertight seal between the interior of the wash cavity 112 and the exterior of the housing 110 can be used.

In another variation, the drain gate 114 is connected to a proximal end of a waste tube, the distal end of the waste tube arranged proximal a drain, to carry waste away from the housing 110 and into the drain (e.g., toilet bowl, shower drain, tub drain, sink).

In yet another variation, the drain gate 114 is connected to a waste reservoir arranged within the housing 110 and configured to receive waste from the wash cavity 112 via the drain gate 114. In one example of this variation, the waste reservoir is removably coupled to the housing 110, allowing the waste reservoir to be emptied and/or cleaned independently of the housing 110.

### 3.11 Drying Element

Generally, the housing 110 includes a drying element 170 (e.g., microwave, conduction, convection, radiation). The drying element 170 can include: a microwave emitter 174 (e.g., a magnetron), a microwave aperture 176 (e.g., waveguide), and a fan 179. The microwave emitter 174 is connected to a proximal end of the microwave aperture 176. The distal end of the microwave aperture 176 is connected to the wash cavity 112. The fan 179 is arranged proximal the microwave emitter 174, opposite the microwave aperture 176 and configured to blow air over the microwave emitter 174 and into the wash cavity 112. The wash cavity 112 is surrounded by a microwave opaque structure to contain the microwaves within the wash cavity 112 once emitted, thereby shielding from the emitted microwaves any sensitive electronics external to the wash cavity 112 and any persons proximal the housing 110 when in operation.

The microwave emitter 174 is configured to emit microwaves into the wash cavity 112 via the microwave aperture 176. The fan 179 generates an airflow over the microwave emitter 174, thereby moving warm air into the wash cavity 112. The microwave emitter 174 receives control signals from a controller 116 in communication with a sensor array (e.g., a humidity sensor, a thermocouple, an infrared sensor). The sensor array can detect the temperature and/or humidity level of the wash cavity 112 and/or the reusable diaper and the controller 116 can infer the water content of the reusable diaper based on data from the sensor array. The controller 116 triggers the microwave emitter 174 to cease emitting microwaves when the temperature and/or humidity level of the wash cavity 112 drops below a threshold, to conserve power and prevent burning and/or other damage to the reusable diaper and the components arranged within the wash cavity 112. In one variation, the microwave emitter 174 is configured to emit microwaves for a duration of time. In one implementation, the microwave emitter 174 receives input from the controller 116, based on feedback data from the sensor array, and regulates microwave generation to optimize drying time while not damaging the reusable diaper, the other components in the housing 110, and/or harming the user.

In example, the drying element 170 of the system 100 is a microwave unit 172. The microwave unit 172 includes a microwave emitter 174 arranged within the housing 110, facing the reusable diaper retained on the rack 130, and configured to dry the reusable diaper. The microwave unit 172 also includes a microwave aperture 176 interposed between the microwave emitter 174 and the wash cavity 112 and configured to direct microwaves toward the reusable diaper.

In another example, the system 100 can include a vent 177 arranged on an exterior face of the housing, an exhaust system 178 coupled to the housing 110, a fan 179 arranged at an outlet of the exhaust system 178, and a controller 116 arranged within the housing 110 and in communication with the fan 179. The controller 116 can, prior to engaging the drying element 170 during a dry cycle, activate the fan 179, draw air through the vent 177 on the top exterior surface of the housing 110, into the wash cavity 112, over the reusable diaper, and out the exhaust system 178.

### 3.12 Controller

In one variation, the system 100 includes a controller 116 electrically connected to and configured to control components of the system 100. In particular, the controller 116 coordinates the various operations of the system 100 by: receiving inputs from the sensors (e.g., temperature, humidity, gas, optical, infrared); processing inputs and a set of pre-loaded logic or computer readable instructions; and controlling the components of the system 100 via electrical signals.

For example, the controller 116 can trigger the spray nozzle 140 to spray the reusable diaper retained on the rack 130 with the second volume of detergent and trigger the brush assembly 150 to sweep the brush head 151 across the reusable diaper, retained on the rack 130, to scrub waste from the reusable diaper downward toward a bottom portion of the wash cavity 113. Further, the controller 116 can trigger the spray nozzle 140 to spray the reusable diaper, retained by the rack 130, with rinse water and activate the drying element 170 to dry the reusable diaper.

In one variation, the system further includes a user interface 117. In this variation, the controller 116 is connected to the user interface 117 and receives control signals (e.g., start, pause, and/or stop processing cycle; repeat a wash, rinse, scrub, and/or dry cycle) from a user via the user interface 117. The controller 116 can derive information the user interface 117 can display to the user before, during, after and between processing cycles.

For example, the system 100 can include a user interface 117 and a controller 116, the controller 116 in electronic communication with the user interface 117. In this example, the controller 116 is configured to: initiate a processing cycle based on a user input received via the user interface 117; derive a processing cycle update based on a processing cycle duration; generate a notification comprising the processing cycle update; display the notification to the user via the user interface 117; display a remaining duration of the processing cycle duration; and display a detergent volume status based on a count of processing cycles completed.

In another variation, the controller 116 includes a wireless communication module. In this variation, the controller 116 transmits data to and/or receives control signals from an external device via the wireless communication module. For example, the controller 116 can interface with wireless communication module: to transmit processing cycle statuses to a user’s mobile device; and to receive start, pause, and/or stop processing cycle commands from the user’s mobile device (e.g., a computer, a cellphone, a tablet, a smartphone).

In another example, the system 100 can include a set of humidity sensors 197 arranged within the exhaust system 178 and configured to output a set of humidity values. The system 100 can also include a set of temperature sensors 198 arranged proximal the set of humidity sensors 197 within the exhaust system 178 and configured to output a set of temperature values. The controller can, during the dry cycle, read the set of humidity values and the set of temperature values from the set of temperature sensors 198 and the set of humidity sensors 197, and in response to the set of humidity values falling below a humidity threshold and the set of temperature values above a temperature threshold, terminate the dry cycle, and disengage the drying element.

In one variation, the system 100 can include an optical sensor 196 (e.g., a camera, an infrared sensor) arranged in the wash cavity. The optical sensor 196 can be configured to have a field of view including the rack retaining the reusable diaper. In one implementation the optical sensor 196 can capture images, allowing the controller implement computer vision techniques to derive: a waste type, a waste level, a diaper condition. The characteristics identified can then be communicated to the user via an associated mobile device or integrated user interface. In another implementation, the optical sensor 196 can output a signal indicating smoke in the wash cavity. Further, the optical sensor 196 can capture an image on which the controller can implement computer vision techniques to derive a drying status of the reusable diaper during the drying cycle. Thus, the controller can receive an image or signal from the optical sensor 196, derive the drying status of the reusable diaper and/or an overheating event and initiate a system shutdown.

### 3.13 Second Housing

In one variation, the system 100 includes a second housing 119 configured to contain components of the system including: the fluid distribution system 160; detergent reservoir 122; controller 116; and/or user interface 117. In one implementation of this variation, the second housing 119 contains the water reservoir 120 and is removably coupled to the housing 110. In another implementation of this variation, the second housing 119 includes storage for additional detergent, additional reusable diapers, or other supplies.

In one example of this variation, the system 100 includes a power supply connection 118, configured to connect to a household power source, and a second housing 119. The system 100 also includes: the second housing 119 coupled to a top surface of a toilet tank; a bottom side of the housing 110 arranged external to the housing 110; and the power supply connection 118, the water reservoir 120, and the detergent reservoir 122 arranged in the second housing 119, the water reservoir 120 and the detergent reservoir 122 fluidly coupled to the spray nozzle 140 in the wash cavity 112 via the fluid distribution system 160.

### 3.14 Support Frame

In one variation, the frame is freestanding over the toilet and does not mount to the wall proximal to the toilet. The frame includes a left lower support structure arranged parallel to and in contact with the floor proximal the toilet and connected at a 90-degree angle to the left vertical support 182. The frame includes a right lower support structure arranged parallel to and in contact with the floor proximal the toilet and connected at a 90-degree angle to the right vertical support 183. The lower support members extend away from the tank of the toilet in the direction of the bowl of the toilet. The lower support members extend a distance greater than the length of the vertical supports to extend the center of gravity of the support structure and housing 110 combination beyond the toilet bowl such that when the housing 110 is placed in the operating position, the frame remains stable and supports the weight of the housing 110. The frame also includes a left lower stabilizing member that extends parallel to and in the opposite direction of the lower left support member (i.e., toward the wall behind the toilet tank) to provide stability to the frame when the housing 110 is placed in the stowed configuration. The frame similarly includes a right stabilization member that extends parallel to and in the opposite direction of the lower right support member. In one example, the frame can couple to and be supported by the toilet, without being in contact with the floor.

In one implementation, the frame is a stationary frame 180 and secures to the wall proximal the toilet. The frame also includes a left arm member 184 and a right arm member 185 pivotably attached to the respective sides of the housing 110 and coupled to the left vertical support 182 and the right vertical support 183 with spring hinges, the spring hinges in conjunction with counterbalances, configured to provide assistance when transitioning the housing 110 between the operational and the stowed positions.

In another variation, the frame is a mobile frame 186, including wheels and enabling the mobile frame 186 and housing 110 to be moved away from the toilet when not in use. The mobile frame 186 includes support members attached to the housing 110 and configured to support the weight of the housing 110 and wheels. The mobile frame 186 includes a void below the housing 110 to receive the toilet when the housing 110 -connected to the mobile frame 186 - is positioned in the operating position above the toilet bowl. The wheels of the mobile frame 186 can define a set of four casters positioned at four lower corners of the mobile frame 186. The water supply connection can include a quick connect fitting at the tee junction to allow the housing 110 to be selectively attached to the household water supply when in use and detached from the household water supply when not in use and moved away from the toilet. Additionally, or alternatively, the water supply connection can include a hose 168 arranged within the toilet tank, and a pump configured to draw water from the toilet tank into the water reservoir 120.

### 3.15 Direct Water Supply

In one variation, the fluid distribution system 160 receives water directly from the household waterline without a pump or water reservoir 120. The household waterline is connected to a valve 164 arranged within the housing 110, where the valve 164precedes the spray nozzle 140 in the path of water through the fluid distribution system 160 and is configured to selectively release water - supplied under pressure by the household waterline - into the wash cavity 112 during the processing cycle, in response to control signals received from the controller 116.

### 3.16 Independent Housing

In one variation, the housing 110 is independent (e.g., without a frame) and is transported by a user from a storage location to the toilet bowl rim for use. The housing 110 includes a feature on the bottom face configured to couple with the toilet bowl rim to hold the housing 110 in a secure position, reducing the likelihood that the housing 110 falls into the toilet bowl or falls off the toilet bowl rim. The user fills the water reservoir 120 manually. The independent housing 110 includes an electrical connection that connects to a wall outlet proximal the toilet to provide power to the housing 110.

### 3.17 Power Supply

In one variation, the housing 110 includes an internal power supply including a set of replaceable batteries. In another variation, the power supply is a removable and rechargeable battery pack. In yet another variation, the power supply is an onboard, non-removable, rechargeable battery.

### 3.18 Water Supply, Fluid Distribution System, and Connections

In one variation, the water supply is a toilet tank fluidly coupled to the fluid distribution system 160 via a water supply connection (e.g., hose 168, pipe, tube). The water supply connection is configured to be low profile and pass from the interior of the toilet tank, under the toilet tank lid, and into the housing 110 without disturbing the natural position of the toilet tank lid on the toilet tank. The water supply connection enables the fluid distribution system 160 to draw water from the toilet tank and pump the water into the water reservoir 120, or directly into the wash cavity 112 via the spray nozzle 140. In one implementation of this variation, the water supply connection includes a weighted distal end opposite the proximal end of the water supply connection fluidly coupled to the fluid distribution system 160; the weighted distal end enabling the distal end of the water supply connection to remain submerged in the water contained within the toilet tank while in operation. In yet another implementation of this variation, the water supply connection is submerged in another water supply volume.

In another variation, the system 100 includes a tank fluid distribution system 160 arranged in the tank of the toilet, a water supply connection from the tank fluid distribution system 160 to the water reservoir 120 in the housing 110, and an electrical connection from the housing 110 to the tank fluid distribution system 160 to provide power and control to the tank fluid distribution system 160. The water supply connection between the housing 110 and the tank fluid distribution system 160 are configured to be low profile and pass from the interior of the toilet tank, under the toilet tank lid, and into the housing 110 without disturbing the natural position of the toilet tank lid on the toilet tank.

In yet another variation, the housing 110 includes a bowl sump snorkel that lowers into toilet bowl to draw water from the toilet bowl into the water reservoir 120. The bowl sump snorkel defines an automatically extending tube that extends from the bottom face of the housing 110 into the toilet bowl, below the water level in the toilet bowl. The extendable tube includes a force sensor that detects resistance to the forward progress of the extension. When resistance is detected by the force sensor, the tube ceases to extend. The fluid distribution system 160 is engaged to draw water from the toilet bowl into the water reservoir 120. When the water level in the water reservoir 120 exceeds a threshold water level, the bowl sump snorkel is retracted back into the housing 110, prior to an initial rinse.

### 3.19 Translating Brush Assembly

In one variation, the articulated brush head 151 support is configured to translate up and down the rack 130 to bring the brush head 151 into contact with the reusable diaper. The articulated brush head 151 support is mounted to a translating brush head 151 carrier and translates along the length of the rack 130. The articulated brush head 151 support can also alter the distance between the brush head 151 and the washing area, more specifically between the flexible bristles and the reusable diaper, by pivoting the articulated brush head 151 support at the connection between the articulated brush head 151 support and the translating brush head 151 carrier.

In a sub-variation of the translating brush assembly 150, the brush head 151 further defines a semi-rigid core to which the flexible bristles are attached. At the conclusion of the rinse cycle, the brush assembly 150 translates to the upper section of the rack 130, and the articulated brush head 151 support brings the semi-rigid core into contact with the reusable diaper - which is swollen with water sprayed during the rinse cycle - such that the reusable diaper deforms, and water is forced out of the absorbent polymer mesh. The translating brush assembly 150 translates downward toward the bottom portion of the rack 130, enabling the brush head 151 to freely rotate, thereby reducing friction between the brush head 151 and the reusable diaper, and reducing the likelihood of tearing the reusable diaper. As the translating brush assembly 150 translates downward, water is forced out of the reusable diaper. By mechanically forcing water out of the reusable diaper via the translating brush assembly 150, the drying time can be reduced, and therefore the power consumed by the drying element 170 can also be reduced.

### 3.20 Pumped Hot Wash + Cold Rinse

In one variation of the system 100, the detergent reservoir 122 is configured to store the second volume of dilute detergent and includes a heating element 124 configured to heat the second volume of dilute detergent, stored in the detergent reservoir 122, to a wash temperature. Further, the fluid distribution system 160 includes: a detergent pump 161 configured to pump heated dilute detergent from the detergent reservoir 122 through the nozzle to wash the reusable diaper, retained on the rack 130, during the processing cycle. The fluid distribution system 160 can also include a water pump 162 configured to pump water from the water reservoir 120 through the nozzle to rinse the reusable diaper, retained on the rack 130, during the processing cycle.

### 3.21 Pumped Hot Wash + Valved Cold Rinse

In one implementation, the system 100 includes a heating element 124 configured to heat the first volume of water, stored in the water reservoir 120, to a wash temperature. Further, the fluid distribution system 160 of the system 100 includes a pump configured to pump heated water from the water reservoir 120 and detergent from the detergent reservoir 122 through the nozzle to wash the reusable diaper, retained on the rack 130, during the processing cycle. The fluid distribution system 160 also includes a valve 164 coupled to a pressurized water supply external to the housing 110 and configured to selectively pass water from the pressurized water supply to the nozzle to rinse the reusable diaper, retained on the rack 130, during the processing cycle.

### 4. Operation & Wash Cycle

Generally, the housing 110 is transitioned from a stowed position into an operating position by the user. The user can open the lid 111, load a soiled reusable diaper, and close the lid 111, triggering the processing cycle to automatically begin. In one variation, the system 100 initiates the wash cycle in response to an input received via the user interface 117. In another variation, the system 100 can receive processing cycle input from the user via the user interface 117 and initiate variations of the processing cycle and/or the subprocesses based on the input received.

### 4.1 Transition From Stowed Position to Operating Position

Generally, to operate the housing 110, a user lowers the housing 110 from the stowed position above the toilet tank, into the operating position proximal the toilet bowl. The user can transition the housing 110 from the stowed position to the operating position with a minimum of effort due to the counterbalanced support members. The frame supports the weight of the housing 110 and keeps the housing 110 stable and in an upright orientation in all positions. The bottom face of the housing 110 is configured to mate with a generic toilet bowl configuration, and rests on the toilet seat or toilet bowl rim in the operating position, with the drain gate 114 of the housing 110 proximal the toilet bowl.

In one example, the system 100 includes a position sensor 194 (e.g., a limit switch, a Hall effect sensor) arranged on an exterior left face of the housing 110 opposite an inner surface of the left arm member 184 in the stowed position and configured to output a position signal representing the housing 110 rests in the stowed position. The system 100 can also include a controller 116 arranged within the housing 110 and in communication with the position sensor 194. The controller 116 can be configured to detect an absence of the position signal. In response to detecting an absence of the position signal, the controller 116 can open the valve 164, allowing the first volume of water to flow into the water reservoir 120. The controller 116 can then, in response to the first volume of water occupying the water reservoir 120: close the valve 164; disengage the fluid distribution system 160; and activate a heating element 124 to heat the first volume of water in the water reservoir 120.

### 4.2 Loading the Housing

Generally, to prior to initiation of a processing cycle, the system 100 receives a soiled reusable diaper into the wash cavity 112 and onto the rack 130. To load the system 100, the user opens the lid 111, and inserts the soiled reusable diaper, through the top of the housing 110, into the wash cavity 112. The user lays the reusable diaper against the top edge of the rack 130, so the clean side of the reusable diaper is in contact with the rack 130 and the soiled side of the reusable diaper is opposite the rack 130, facing the spray nozzle 140, brush assembly 150, and drying element 170. The set of clips 132 engages the top edge of the reusable diaper at the top edge of the rack 130, retaining the reusable diaper on the rack 130. The processing cycle can initiate when the lid 111 is closed

In one variation, the user may initiate the processing cycle by interfacing with a control panel arranged on the exterior of the housing 110. The user may select a specific wash cycle or select other settings such as an option to start the processing cycle at a preselected time, water temperature, detergent quality, duration of processing cycle, duration of specific sub processes, etc.

In one example, the system 100 can include a lid 111 arranged over the wash cavity 112, a latch 192 configured to secure the lid 111 in a closed position during the processing cycle, an optical sensor 196 196 (e.g., camera, infrared) defining a field of view facing outwardly from the housing, and a controller 116 arranged within the housing 110, in communication with the optical sensor 196 196. The controller can access an image captured by the optical sensor 196 196 scan the image for an optical fiducial identifying the reusable diaper, detect the optical fiducial in the image, activate a heating element 124 to heat the volume of water occupying the water reservoir 120, trigger the latch 192 to open the lid 111, and, in response to closure of the lid 111, initiate the processing cycle.

In another example, the system 100 can also include a lid 111 arranged over the wash cavity 112, an optical sensor 196 196 defining a field of view facing the rack 130, and a controller 116. The controller 116 can be configured to, at a first time and in response to closure of the lid 111, trigger the optical sensor 196 196 to capture a first image, scan the first image for an optical fiducial, and in response to detecting the optical fiducial in a target region in the first image: confirm the presence of the reusable diaper retained on the rack 130 and activate the processing cycle. Further, the controller 116 can be configured to, at a second time and in response to closure of the lid 111, trigger the optical sensor 196 196 to capture a second image scan the second image for a second optical fiducial. The controller 116 can then, in response to detecting an absence of the second optical fiducial in a target region in the second image, detect misalignment of a second reusable diaper and generate a prompt to reload the second reusable diaper on the rack 130.

### 4.3 Filling the Water Reservoir

Once the processing cycle initiates, water is drawn from the household water supply into the water reservoir 120 via the fluid distribution system 160. A volume of water sufficient to complete the processing cycle (e.g., to dislodge solid waste, wash the reusable diaper via agitation, rinse the reusable diaper, and rinse the wash cavity 112 after extraction of the reusable diaper, etc.) is drawn into the water reservoir 120.

In one variation, once the housing 110 is in the operating position, water is drawn from the water supply into the water reservoir 120 while the user loads the reusable diaper into the housing 110. In response to the lid 111 closing and retention of the reusable diaper on the rack 130 with the set of clips 132, the controller 116 can initiate the processing cycle.

### 4.4 Initial Rinse and Dislodgement of Solid Waste

Generally, the first step of the processing cycle dislodges solid waste from the soiled reusable diaper. The drain gate 114 opens, exposing the interior of the wash cavity 112 to the toilet bowl proximal and below the housing 110. The fluid distribution system 160 draws water from the water reservoir 120 and sprays the water onto the reusable diaper to dislodge solid waste via the spray nozzle 140.

The spray nozzle 140 is configured to spray in a pattern that impacts the full length and the full width of the reusable diaper with enough velocity (provided by the water pressure supplied by the fluid distribution system 160) to dislodge solid waste from the reusable diaper. The spray nozzle 140, arranged in the upper section of the wash cavity 112, generally directs the water towards the lower region of the wash cavity 112 proximal the drain gate 114. The spray nozzle 140 initially aims at the upper edge of the rack 130, proximal the set of clips 132. The nozzle slowly pivots downward and stops pivoting when the spray is proximal or slightly past the bottom edge of the rack 130, the spray dislodging solid waste from the reusable diaper throughout the pivot period.

The dislodged solid waste travels toward the bottom portion of the wash cavity 113 under the combined force of the water stream and the force of gravity. The dislodged waste vacates the wash cavity 112 via the drain gate 114 and falls into the toilet bowl below. In response to completion of the initial rinse cycle, the drain gate 114 is closed, creating a watertight wash cavity 112. In one variation, the water stream continues until a sensor (e.g., optical) detects no remaining solid waste, or the level of solid waste detected is below a threshold amount.

For example, the fluid distribution system 160 can include: a valve 166 interposed between the wash cavity 112 and the drain gate 114; and a controller 116. The controller 116 can be configured to trigger the valve 166 to close at a first time during a wash cycle, in the processing cycle, to collect water and detergent in the wash cavity 112. The controller 116 can also: trigger the valve 166 to open at a second time to release water, detergent, and waste from the wash cavity 112 into the toilet bowl via the drain gate 114 during the wash cycle; and release water from the wash cavity 112 into the toilet bowl via the drain gate 114 during a rinse cycle succeeding the wash cycle in the processing cycle.

### 4.5 Wash Cycle

Generally, after the initial rinse cycle and dislodgement of solid waste, the system 100 performs a wash cycle, applying detergent to the soiled reusable diaper to enhance waste removal. During the wash cycle, a volume of water and detergent is dispensed into the wash cavity 112, collected by a sump in the bottom portion of the wash cavity 113, and recirculated through the spray nozzle 140.

The fluid distribution system 160 pumps detergent from the detergent reservoir 122 and sprays the detergent onto the reusable diaper, coating the soiled side of the reusable diaper, via the spray nozzle 140. Additionally or alternatively, the spray nozzle 140 can spray a water-detergent solution onto the reusable diaper. In one variation, a set of pumps 169 fluidly coupled to the detergent reservoir 122, the water reservoir 120, and the spray nozzle 140 - via the fluid distribution system 160 - draw water and detergent from the respective reservoirs and spray the water and detergent, via the spray nozzle, onto the reusable diaper. The water and detergent sprayed in a ratio of detergent to water that optimizes waste removal while minimizing consumption. A sump assembly can retain gathered washing liquid after the washing liquid contacts the reusable diaper.

In one example, the system 100 includes a pump fluidly coupled to the water reservoir 120, the detergent reservoir 122, and the spray nozzle 140. The pump can be configured to pump the first volume of water from the water reservoir 120 and the second volume of dilute detergent from the detergent reservoir 122, to the spray nozzle 140. The first volume of water and the second volume of detergent can then be sprayed under pressure, onto the reusable diaper, via the spray nozzle 140.

The fluid distribution system 160 is coupled to the sump via a sump valve arranged between the sump and the fluid distribution system 160. During the wash cycle, the sump valve is opened, and the fluid distribution system 160 draws the water-detergent solutions from the sump and re-circulates it to the spray nozzle 140 to again be sprayed onto the reusable diaper. The sump valve includes a filter on the sump side that limits the size and/or number of solid waste particles entering the fluid distribution system 160 and/or spray nozzle 140, reducing the likelihood that the fluid distribution system 160, spray nozzle 140, or connections become obstructed by solid waste suspended in the water-detergent solution.

In another example, the system 100 includes a sump assembly arranged within the wash cavity 112. The sump assembly includes: a sump, arranged in a bottom portion of the wash cavity 113; a sump filter; and a sump pump. The sump assembly can be configured to recycle filtered wastewater from the sump to the spray nozzle 140 via the fluid distribution system 160.

In one variation, a valve (e.g., venturi, capillary) in the fluid distribution system 160 selectively allows detergent from the detergent reservoir 122 to join the flow of water from the water reservoir 120 to the spray nozzle 140, to produce a detergent and water solution. However, any suitable method of introducing detergent into the water supply to the spray nozzle 140 can be used. In another variation, the detergent is injected directly into the wash cavity 112 via a detergent nozzle.

### 4.6 Scrub Cycle

During the scrub cycle, succeeding the wash cycle, the rack 130 can translate toward the bottom portion of the housing 110, submerging the reusable diaper in the cleaning liquid in the bottom portion of the housing 110. The brush head 151 can then rotate downward while the brush assembly 150 positions the brush head 151 proximal to the reusable diaper and brings the flexible bristles of the brush head 151 into contact with the soiled side of the reusable diaper. The rack 130 can translate upward and downward to repeatedly bring the soiled side of the reusable diaper into contact with the bristles of the brush head 151. The rotating brush head 151 agitates the reusable diaper and, in cooperation with the water-detergent solution, dislodges any further waste adhered to and embedded in the reusable diaper. As the brush head 151 rotates, the flexible bristles push any dislodged waste downward toward the bottom portion of the wash cavity 113. In one variation, after a preselected scrub cycle duration, the brush assembly 150 returns to an original position in the wash cavity 112, the brush head 151 ceases rotation, the rack 130 translates up, removing the reusable diaper from the water-detergent solution, and the sump valve closes. In one variation, in response to a sensor (e.g., an optical sensor 196) detecting an amount of waste on the reusable diaper less than a threshold amount, the brush head 151 ceases rotation and the rack 130 translates up and out of the water-detergent solution.

For example, the system 100 can include a controller 116, a rack actuator 136, and a linear track 138, the linear track 138 coupled to the rack 130. In this example, the controller 116 can trigger the rack actuator 136 to raster the rack 130 from a first position below the brush head 151 to a second position above the brush head 151 along the linear track 138, the brush head 151 in contact with the reusable diaper retained by the rack 130, rotating in a downward direction brushing waste toward the drain gate 114.In another example, the system 100 can include: a controller 116; a brush actuator 153; a gear rack 154; a gearbox 155; and a brush head gear 151. The gearbox 155 is configured to: engage the gear rack 154; turn the brush head gear 151; and rotate the brush head 151 downward, the brush head 151 in contact with the reusable diaper retained by the rack 130. The controller 116 is configured to trigger the brush actuator 153 to raster the brush head 151 from a first position above the rack 130 to a second position below the rack 130, along the gear rack 154, rotate the brush head 151 downward, brushing waste toward the drain gate 114. In response to the brush head 151 reaching the second position below the rack 130, the controller 116 can engage the brush actuator 153 to raster the brush head 151 to a third position, below the second position, the brush head 151 submerged in a volume of waste liquid occupying a bottom portion of the wash cavity 113. The controller 116 can then, in response to reaching the third position, engage the brush actuator 153 rotate the brush head 151 to dislodge waste accumulated on the brush head 151 into the volume of waste liquid and raster the brush head 151 from the third position to the first position above the rack 130, along the gear rack 154.

### 4.7 Rinse Cycle

During the rinse cycle, succeeding the scrub cycle, the drain gate 114 opens, and the cleaning liquid and any waste suspended in the liquid flows from the wash cavity 112 and into the toilet bowl positioned below the housing 110. The fluid distribution system 160 draws water from the water reservoir 120 and pumps the water through the spray nozzle 140. The spray nozzle 140 pivots upward and downward to rinse the reusable diaper. After contact with the reusable diaper, the rinse water flows through the open drain gate 114 into the toilet bowl below. The spray nozzle 140 continues to rinse the reusable diaper for a rinse cycle duration. At the expiration of the rinse cycle duration, the spray nozzle 140 ceases to rinse the reusable diaper. The drain gate 114 can close after a pause to allow any excess water to drain from the wash cavity 112.

### 4.8 Dry Cycle

The drain gate 114, in addition to providing a watertight seal, includes electromagnetic shielding. Therefore, when closed during the dry cycle, the drain gate 114 prevents microwaves from escaping the wash cavity 112. The microwave emitter 174 emits microwaves into the wash cavity 112 of the housing 110 for a duration of time or until a humidity sensor 197 detects that a humidity level of the wash cavity 112 falls below a threshold humidity level. The fan 179 generates an airflow over the microwave emitter 174 during the dry cycle in the wash cavity 112. The airflow circulates heated air through the wash cavity 112, enabling further drying of the reusable diaper, and carries steam emitted by the reusable diaper and humid air out of the wash cavity 112 through a vent 177. Steam produced during the drying phase is carried by the airflow generated by the fan 179 and out through the vent 177. In one variation, a temperature sensor 198 detects a temperature of the diaper and/or of the air in the wash cavity 112. A controller 116 then signals the microwave emitter 174 to restrict microwave emissions to maintain temperatures at or below a safety and efficiency temperature threshold, based on the temperature data provided by the temperature sensor.

### 4.9 Removal of Clean Reusable Diaper

At the conclusion of the dry cycle, an indicator arranged in the housing 110 can emit an audible and/or visual signal indicating that the dry cycle is complete, and that it is safe for a user to open the lid 111 of the housing 110. The user can then open the lid 111, remove the reusable diaper from the rack 130 in the wash cavity 112 and return the lid 111 to a closed position.

### 4.10 Final Rinse and Sanitation Cycle

Once the lid 111 is in a closed position, the drain gate 114 can open and the fluid distribution system 160 can draw water from the water reservoir 120 and pump it to the spray nozzle 140. The spray nozzle 140 sprays water to rinse the interior of the wash cavity 112, pivoting upwards and downwards to direct the water stream to all surfaces within the wash cavity 112.

The final rinse water flows into the toilet bowl below. The drain gate 114 then closes. The microwave emitter 174 can emit microwaves for a duration of time to evaporate the water in the interior of the wash cavity 112. The housing 110 emits an audible and/or visual signal indicating the housing 110 is now dry and ready to be transitioned to the stowed position or initiate a new processing cycle.

In one variation, in which the system 100 includes a sanitizing solution reservoir, a sanitizing solution is injected into the water before reaching the spray nozzle 140. The sanitizing solution disinfects the interior surfaces of the wash cavity 112 and various components within the wash cavity 112. In another variation, the microwave emitter 174 ceases to emit microwaves when a humidity sensor 197 detects the humidity level within the wash cavity 112 drops below a threshold level.

### 4.11 Stowing the Housing

After the completion of the final rinse and sanitation cycle of the final processing cycle, the housing 110 transitions from the operating position to the stowed position, enabling access to the toilet. Due to the counterbalanced support members, a user may transition the housing 110 from the operating position to the stowed position by exerting minimal force. Once in the stowed position, the user powers off the housing 110.

In one variation, the housing 110 automatically powers on when transitioned into the operation position and automatically powers off when transitioned into the stowed position.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A system of washing and drying a reusable diaper comprising:
a housing defining:
a wash cavity; and
a drain gate configured to face a toilet bowl;
a water reservoir:
arranged within the housing; and
configured to store a first volume of water;
a detergent reservoir:
arranged within the housing; and
configured to store a second volume of dilute detergent;
a rack:
arranged within the wash cavity; and
configured to support and retain the reusable diaper during a processing cycle;
a spray nozzle:
arranged within the wash cavity; and
◯configured to spray water, from the water reservoir, toward the reusable diaper retained on the rack;
a brush assembly:
arranged within the wash cavity;
comprising a brush head; and
configured to sweep the brush head across the reusable diaper retained on the rack;
a fluid distribution system:
arranged within the housing; and
configured to:
distribute water from a water supply into the water reservoir;
distribute water from the water reservoir to the spray nozzle;
distribute detergent from the detergent reservoir onto the reusable diaper retained on the rack; and
release soiled water into the toilet bowl via the drain gate; and
a drying element:
arranged within the housing; and
configured to dry the reusable diaper retained on the rack.

2. The system of claim 1:
wherein the detergent reservoir is configured to store the second volume of dilute detergent;
further comprising a heating element configured to heat the second volume of dilute detergent, stored in the detergent reservoir, to a wash temperature; and
wherein the fluid distribution system comprises:
a detergent pump configured to pump heated dilute detergent from the detergent reservoir through the nozzle to wash the reusable diaper, retained on the rack, during the processing cycle; and
a water pump configured to pump water from the water reservoir through the nozzle to rinse the reusable diaper, retained on the rack, during the processing cycle.

3. The system of claim 1:
further comprising a heating element configured to heat the first volume of water, stored in the water reservoir, to a wash temperature; and
wherein the fluid distribution system comprises:
a pump configured to pump heated water from the water reservoir and detergent from the detergent reservoir through the nozzle to wash the reusable diaper, retained on the rack, during the processing cycle; and
a valve:
coupled to a pressurized water supply external to the housing; and
configured to selectively pass water from the pressurized water supply to the nozzle to rinse the reusable diaper, retained on the rack, during the processing cycle.

4. The system of claim 1:
wherein the fluid distribution system comprises a valve interposed between the wash cavity and the drain gate; and
further comprising a controller, configured to:
trigger the valve to close at a first time during a wash cycle, in the processing cycle, to collect water and detergent in the wash cavity; and
trigger the valve to open at a second time to:
release water, detergent, and waste from the wash cavity into the toilet bowl via the drain gate during the wash cycle; and
release water from the wash cavity into the toilet bowl via the drain gate during a rinse cycle succeeding the wash cycle in the processing cycle.

5. The system of claim 1, further comprising a controller, configured to:
trigger the spray nozzle to spray the reusable diaper retained on the rack with the second volume of detergent;
trigger the brush assembly to sweep the brush head across the reusable diaper, retained on the rack, to scrub waste from the reusable diaper toward a bottom portion of the wash cavity;
trigger the spray nozzle to spray the reusable diaper, retained by the rack, with rinse water; and
activate the drying element to dry the reusable diaper.

6. The system of claim 1:
wherein the rack defines:
a perforated panel inclined within the wash cavity and configured to locate a soiled side of the reusable diaper facing the spray nozzle and the brush head;
further comprising a set of clips:
arranged on the rack; and
configured to retain a top edge of the reusable diaper on the rack; and
wherein the brush assembly is configured to:
traverse downwardly, proximal the rack, to scrub waste downward;
maintain contact between the brush head and the soiled side of the reusable diaper; and
tension the diaper downwardly against the set of clips during a scrub cycle in the processing cycle.

7. The system of claim 1:
wherein the drying element comprises a microwave unit comprising:
a microwave emitter:
arranged within the housing;
facing the reusable diaper retained on the rack; and
configured to dry the reusable diaper; and
a microwave aperture:
interposed between the microwave emitter and the wash cavity; and
configured to narrow a field of view of the microwave emitter and to direct microwaves toward the reusable diaper retained on the rack.

8. The system of claim 1:
wherein the water supply comprises a pressurized residential waterline:
fluidly coupled to the fluid distribution system via a valve; and
configured to supply the fluid distribution system with the first volume of water; and
further comprising a stationary frame;
comprising:
a left vertical support vertically positioned on a left side of the toilet;
a right vertical support vertically positioned on a right side of the toilet;
a left arm member pivotably attached to a left side of the housing and coupled to the left vertical support; and
a right arm member pivotably attached to a right side of the housing and coupled to the right vertical support; and
configured to:
support the housing in a stowed position above the tank of toilet; and enable the housing to transition to an operating position above the opening of the toilet bowl.

9. The system of claim 8:
further comprising:
a position sensor:
arranged on an exterior left face of the housing opposite an inner surface of the left arm member in the stowed position; and
configured to output a position signal representing the housing rests in the stowed position; and
a controller:
arranged within the housing;
in communication with the position sensor;
wherein the controller is configured to:
detect an absence of the position signal;
in response to detecting an absence of the position signal, open the valve, allowing the first volume of water to flow into the water reservoir;
in response to the first volume of water occupying the water reservoir:
close the valve;
disengage the fluid distribution system; and
activate a heating element to heat the first volume of water in the water reservoir.

10. The system of claim 1, further comprising a mobile frame:
comprising:
a first set of vertical supports vertically positioned on a left side of the toilet;
a second set of vertical supports vertically positioned on the right side of the toilet;
a left arm member pivotably attached to the left side of the housing and coupled to the first set of vertical supports;
a right arm member pivotably attached to the right side of the housing and coupled to the second set of vertical supports; and
a set of casters, coupled to the first set of vertical supports and the second set of vertical supports, configured to allow movement of the system between a first toilet and a second toilet; and
configured to:
support the housing in a stowed position above the tank of toilet; and
enable the housing to transition to an operating position above the opening of the toilet bowl.

11. The system of claim 10:
wherein the water supply comprises a hose fluidly coupled to the water reservoir; and
further comprising a set of pumps configured to:
draw water, via the hose, from the toilet tank to the water reservoir;
fill the water reservoir with the first volume of water; and
pump water from the water reservoir to the spray nozzle.

12. The system of claim 1, further comprising:
An actuator;
a gear rack;
a brush gear;
a gearbox configured to:
engage the gear rack;
turn the brush gear; and
rotate the brush head downward, the brush head in contact with the reusable diaper retained by the rack; and
a controller configured to trigger the actuator to:
raster the brush head from a first position above the rack to a second position below the rack, along the gear rack;
rotate the brush head downward, to brush waste toward the drain gate;
in response to the brush head reaching the second position below the rack, raster the brush head to a third position, below the second position, the brush head submerged in a volume of waste liquid occupying a bottom portion of the wash cavity;
in response to reaching the third position, rotate the brush head to dislodge waste accumulated on the brush head into the volume of waste liquid; and
raster the brush head from the third position to the first position above the rack, along the gear rack.

13. The system of claim 1 further comprising:
an actuator; and
a linear track, coupled to the rack; and
a controller configured to trigger the actuator to:
raster the rack from a first position below the brush head to a second position above the brush head along the linear track, the brush head in contact with the reusable diaper retained by the rack; and
rotate the brush head in a downward direction to brush waste toward the drain gate.

14. The system of claim 1, further comprising:
a lid arranged over the wash cavity;
a latch configured to secure the lid in a closed position during the processing cycle;
an optical sensor 196 defining a field of view facing outwardly from the housing; and
a controller arranged within the housing, in communication with the optical sensor 196, and configured to:
access an image captured by the optical sensor 196;
scan the image for an optical fiducial identifying the reusable diaper;
in response to detecting the optical fiducial in the image;
activate a heating element to heat the volume of water occupying the water reservoir;
trigger the latch to open the lid; and
in response to closure of the lid, initiate the processing cycle.

15. The system of claim 1, further comprising:
a lid arranged over the wash cavity;
an optical sensor 196 defining a field of view facing the rack;
a controller:
configured to, in response to closure of the lid, at a first time:
trigger the optical sensor 196 to capture a first image;
scan the first image for an optical fiducial; and
in response to detecting the optical fiducial in a target region in the first image:
confirm the presence of the reusable diaper retained on the rack; and
activate the processing cycle; and
configured to, in response to closure of the lid, at a second time
trigger the optical sensor 196 to capture a second image;
scan the second image for a second optical fiducial; and
in response to detecting an absence of the second optical fiducial in a target region in the second image:
detect misalignment of a second reusable diaper; and
generate a prompt to reload the second reusable diaper on the rack.

16. The system of claim 1:
further comprising:
a vent arranged on an exterior face of the housing;
an exhaust system coupled to the housing;
a fan arranged at an outlet of the exhaust system; and
a controller arranged within the housing and in communication with the fan; and
wherein, prior to engaging the drying element during a dry cycle, the controller is configured to:
activate the fan, drawing air through the vent on the top exterior surface of the housing, into the wash cavity, over the reusable diaper, and out the exhaust system.

17. The system of Claim •o▪•:
further comprising:
a set of humidity sensors:
arranged within the exhaust system; and
configured to output a set of humidity values;
a set of temperature sensors:
arranged proximal the set of humidity sensors within the exhaust system; and
configured to output a set of temperature values; and
wherein the controller is configured to, during the dry cycle:
read the set of humidity values and the set of temperature values from the set of temperature sensors and the set of humidity sensors;
in response to the set of humidity values falling below a humidity threshold and the set of temperature values above a temperature threshold, terminate the dry cycle, and disengage the drying element.

18. A system of washing and drying a reusable diaper comprising:
a housing defining:
a wash cavity; and
a drain gate configured to face a toilet bowl;
a stationary frame:
coupled to the housing;
comprising a set of vertical supports vertically positioned on a right side and a left side of a toilet; and
configured to support a full weight of the housing and mount to a wall;
a water reservoir:
arranged within the housing; and
configured store a first volume of water;
a detergent reservoir:
arranged within the housing; and
configured to store a second volume of dilute detergent;
a rack:
arranged within the wash cavity; and
configured to support and retain the reusable diaper during a processing cycle;
a spray nozzle:
arranged within the wash cavity; and
configured to spray water, from the water reservoir, toward the reusable diaper retained on the rack;
a brush assembly:
arranged within the wash cavity;
comprising a brush head; and
configured to sweep the brush head across the reusable diaper retained on the rack;
a fluid distribution system:
arranged within the housing; and
configured to:
distribute water from a household waterline into the water reservoir;
distribute water from the water reservoir to the spray nozzle;
distribute detergent from the detergent reservoir onto the reusable diaper retained on the rack; and
release soiled water into the toilet bowl via the drain gate;
a valve:
fluidly coupled to a household waterline; and
configured to:
in an open position, allow flow of the first volume of water to the water reservoir;
in a closed position, restrict flow of the first volume of water to the water reservoir; and
a drying element:
arranged within the housing; and
configured to dry the reusable diaper retained on the rack.

19. The system of claim 18, further comprising a pump:
fluidly coupled to the water reservoir, the detergent reservoir, and the spray nozzle; and
configured to pump the first volume of water from the water reservoir and the second volume of dilute detergent from the detergent reservoir to the spray nozzle, the first volume of water and the second volume of detergent sprayed under pressure onto the reusable diaper via the spray nozzle.

20. A system of washing and drying a reusable diaper comprising:
a housing defining:
a wash cavity; and
a drain gate configured to face a toilet bowl;
a mobile frame:
configured to position the housing over the opening of the toilet bowl; and
comprising:
a set of vertical supports vertically positioned on a left side and a right side of a toilet; and
a set of casters, coupled to the set of vertical supports, configured to allow movement of the system between a first toilet and a second toilet;
a detergent reservoir:
arranged within the housing; and
configured to store a second volume of dilute detergent;
a rack:
arranged within the wash cavity; and
configured to support and retain the reusable diaper during a processing cycle;
a spray nozzle:
arranged within the wash cavity; and
configured to spray water toward the reusable diaper retained on the rack;
a brush assembly:
arranged within the wash cavity;
comprising a brush head;
configured to sweep the brush head across the reusable diaper retained on the rack;
a fluid distribution system:
arranged within the housing;
comprising a set of pumps; and
configured to:
draw water, via a hose, from a toilet tank to the spray nozzle; and
distribute detergent from the detergent reservoir onto the reusable diaper retained on the rack; and
a drying element:
arranged within the housing; and
configured to dry the reusable diaper retained on the rack.
